(19) United States
(12) Patent Application Publication
(10) Pub. No.: US 2019/0242862 A1
(43) Pub. Date: Aug. 8, 2019

(54) FOOD FRESHNESS SAFETY INDICATOR

(71) Applicant: U.S. Government as Represented by the Secretary of the Army, Natick, MA (US)

(72) Inventors: Greg Burnham, Natick, MA (US); Mark Buller, Douglas, MA (US); Bethany Buller, Douglas, MA (US); Richard Cook, Worcester, MA (US); Daniel Cook, Worcester, MA (US); Theresa Finch, Harrisville, RI (US); Peter Finch, Harrisville, RI (US); Sarah Stevens, Mendon, MA (US); Timothy Mesite, Douglas, MA (US)

(73) Assignee: U.S. Government as Represented by the Secretary of the Army

(21) Appl. No.: 15/677,078

(22) Filed: Aug. 15, 2017

Related U.S. Application Data
(63) Continuation of application No. 14/077,973, filed on Nov. 12, 2013, now abandoned.
(60) Provisional application No. 61/725,512, filed on Nov. 13, 2012.

(51) Int. Cl. G01N 33/02

(52) U.S. Cl. CPC G01N 33/02

(57) ABSTRACT

A system for determining food safety includes a recording device printed with one or more inks configured to change characteristics over predetermined time intervals, and a scanning device configured to extract encoded information from the recording device and convey the extracted information to an analysis service. The analysis service is configured to provide predictive modeling tools for analysis of the extracted information and provide at least one assessment of a food product bearing the recording device to the scanning device.

### STATEMENT OF GOVERNMENT INTEREST

The invention described herein may be manufactured and used by or for the U.S. Government for governmental purposes without the payment of any royalties thereon or therefor.

### FIELD

The disclosed embodiments relate in general to food production, and more particularly to providing an indication of food safety.

### BACKGROUND

The USDA cites improper food handling as the number one cause in an estimated 48 million US cases of food-borne illness each year. While consumer product safety and quality information is provided, it is generally limited to dates designated as “product best by” or “product use by” which are usually established under ideal storage conditions and do not take into account dynamic temperature environments. To combat this problem, food processors, regulators, and academicians utilize a number of predictive modeling tools to evaluate microbial responses demonstrating lag, growth, survival, or inactivation in response to changes in the environment, in particular, temperature changes over time. Upon providing these models with an identification of the food and an accurate time and temperature profile, the models will predict bacteria growth and the risk posed by the toxins they produce. While these tools can provide an accurate assessment of the risk of food borne pathogen behavior, these bacterial behavior models require knowledge of the type of food and the time and temperature profile related to how the food item has been handled.

Existing time and temperature tracking devices for recording temperature changes over time are available in a variety of forms, such as electronic data loggers or color changing stickers. However, data loggers are too expensive to integrate into individual product packaging, lack the ability to readily transfer logged information to a pathogen prediction model, and lack the ability to provide, at a point of purchase, results from the pathogen predictive model specific to an individual package. Color changing stickers are available that use non-reverting temperature sensitive inks that permanently change color when pre-designed time and temperature thresholds are exceeded. These stickers might offer an inexpensive indication as to whether food has been exposed to extreme temperatures, however, they provide only a rough guide to the state of the food and are not capable of capturing and providing information for use with pathogen prediction models, and also lack the ability to provide results specific to an individual package at a point of purchase.

### SUMMARY

The disclosed embodiments are directed to providing food safety information directly to consumers by recording the temperature of the food over time from the point of packaging, providing the recorded data to pathogen prediction models, and conveying the resulting predictions at any time after the food leaves the point of packaging. A system for determining food safety includes a recording device printed with one or more inks configured to change characteristics over predetermined time intervals, and a scanning device configured to extract encoded information from the recording device and convey the extracted information to an analysis service. The analysis service is configured to provide predictive modeling tools for analysis of the extracted information and provide at least one assessment of a food product bearing the recording device to the scanning device.

### BRIEF DESCRIPTION OF THE DRAWINGS

Non-limiting aspects and other features of the disclosed embodiments are explained in the following description and illustrated in the accompanying drawings, wherein:

FIG. 1 shows a block diagram of a system according to the disclosed embodiments;

FIGS. 2A-2B illustrate exemplary reports that may be provided according to the disclosed embodiments;

FIGS. 3A-3C show exemplary illustrations according to the disclosed embodiments; and

FIG. 4 illustrates an exemplary procedure according to the disclosed embodiments.

### DETAILED DESCRIPTION

The disclosed embodiments are generally directed to encoding identification and time based information into a graphic that can be readily scanned and provided to an analysis service that responds with results of the analysis. In one or more aspects, the identification and time based information may be related to a food product and a time and temperature profile to which the food has been exposed. In at least one aspect, the analysis service provides product safety information specific to the identified food based on a pathogen behavior model.

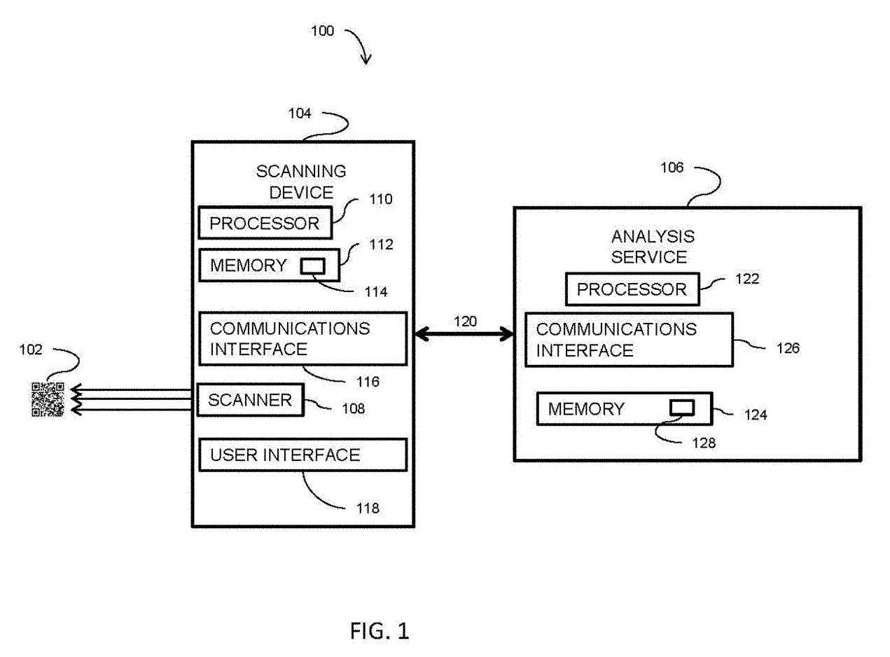

FIG. 1 shows a block diagram of a system 100 according to the disclosed embodiments. System 100 includes a recording device 102, a scanner 104 and an analysis service 106. Recording device 102 generally includes scannable graphics that are capable of encoding information, for example, environmental conditions at set time points, and static information related to the product to which the recording device is attached. It should be understood that the information capable of being recorded and encoded by recording device 102 is not limited and may include any environmental conditions at set time points, for example, temperature, humidity, light, pressure, ambient atmospheric content or any other suitable conditions. It should also be understood that the recording device may be encoded with additional information including manufacturer identification, manufacturing date, manufacturing location, packaging date, batch numbers, ingredient information, ingredient supplier information, identification of a product to which the recording device is attached, or any other suitable information related to the product bearing the recording device. In at least one aspect, the recording device may be encoded with a location to which the recorded information is to be forwarded.

In at least one aspect, the recording device is a tag or label which may be affixed to a product. The graphic may include one or more encoding formats, for example, one or two dimensional barcodes, or any set of symbols or representations for communicating information. At least a portion of the label may be printed with one or more inks that are capable of changing characteristics over time. In at least one aspect, the one or more inks may be programmable such that specific characteristics may change at specific time intervals, certain environmental conditions, or both. For example, the inks may be thermo-chromatic and may change color, become visible, or become transparent, based on a time and temperature exposure. The inks may have other characteristics that may change based on a time and temperature exposure, for example, magnetic, optical, electrical, or electromagnetic characteristics. As a result, a pattern of the scannable graphics may change over time, based on the temperature experienced by the product bearing the recording device. In one or more aspects, a temperature pattern may be locked or encoded by the ink at each of a set of time points. As a result, a time and temperature profile may be recorded that includes temperatures for different time periods. In one exemplary aspect, temperatures for different time periods are recorded within different portions of the graphic. As mentioned above, the graphic may include additional information related to an attached product, in this example including a date of manufacture, batch numbers, suppliers, and a product identifier. The graphic may be scanned at any time after being affixed to the product in order to obtain an environmental profile to which the product has been exposed up to the point of scanning.

The system 100 may also include a scanning device 104. The scanning device 104 at least includes a scanner 108 for scanning the recording device 102 and extracting the encoded information from the recording device 102. The scanner 108 may be capable of recognizing information encoded on the recording device using magnetic, optical, electrical, electromagnetic, or any other technology. In at least one aspect, the scanning device 104 includes a processor 110 and a memory 112 for operating the scanner 108 and processing the encoded information. The memory 112 may include at least one computer readable medium 114 on which may be stored computer readable program code for carrying out and executing the processes described herein. In one or more aspects, the scanning device 104 may be a mobile phone and the scanner 108 may be a camera. The computer readable medium 114 may include one or more scanner applications for decoding the information received from the recording device. For example, when the recording device is in the format of a 2-dimensional bar code referred to as a Quick Response Code (QR code) any number of QR scanning applications may be used to decode the recording device information. The scanning device may also include a communications interface 116 for communication with other devices and a user interface 118 for receiving input and providing output.

The system 100 may further include an analysis service 106 connected to the scanning device 104 by a link 120. The analysis service may include a processor 122, memory 124, and a communications interface 126 for communication with other devices, for example scanning device 104. Similar to memory 112, memory 124 may also include at least one computer readable medium 128 on which may be stored computer readable program code for carrying out and executing the processes described herein. In at least one aspect, the computer readable medium 128 may include applications for analyzing the information extracted from the recording 102 by the scanner 108 and conveyed to the analysis service 106 through link 120. In another aspect, link 120 may include any suitable link for conveying information, including but not limited to a wired or wireless link, a local area or wide area network, the Internet, or any other suitable link for conveying information between the scanning device 104 and the analysis service 106.

In at least one exemplary aspect, the analysis service may provide predictive modeling tools for analysis of the information extracted by the scanning device 104. In one or more aspects, the predictive modeling tools may include models that will predict bacteria growth in response to temperature changes over time in food products. The information extracted from the recording device may include identification of the food product and a time and temperature profile to which the food product was exposed up to the point of scanning the recording device, and the models may predict bacteria growth, an amount of resulting toxins and an assessment of the associated risks.

In at least one aspect, the analysis service 106 may be incorporated in scanning device 104 while in other aspects the analysis service 106 may be distributed among any number of computing devices. In further aspects, the analysis service may be accessible through a URL over the Internet.

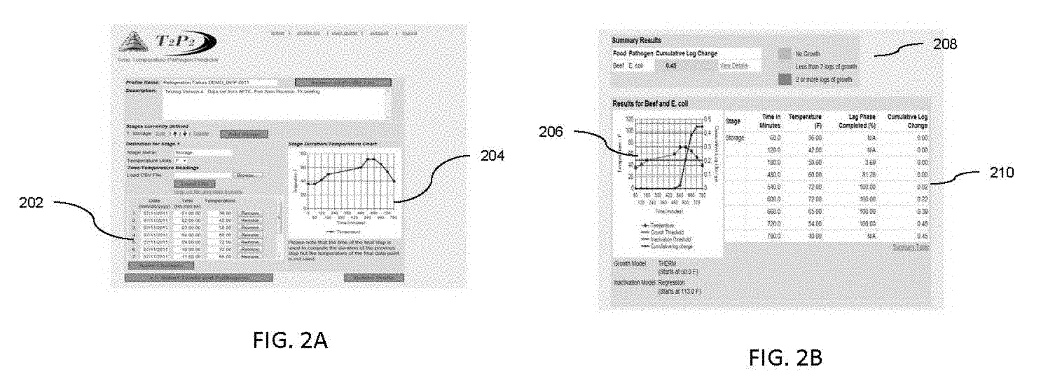

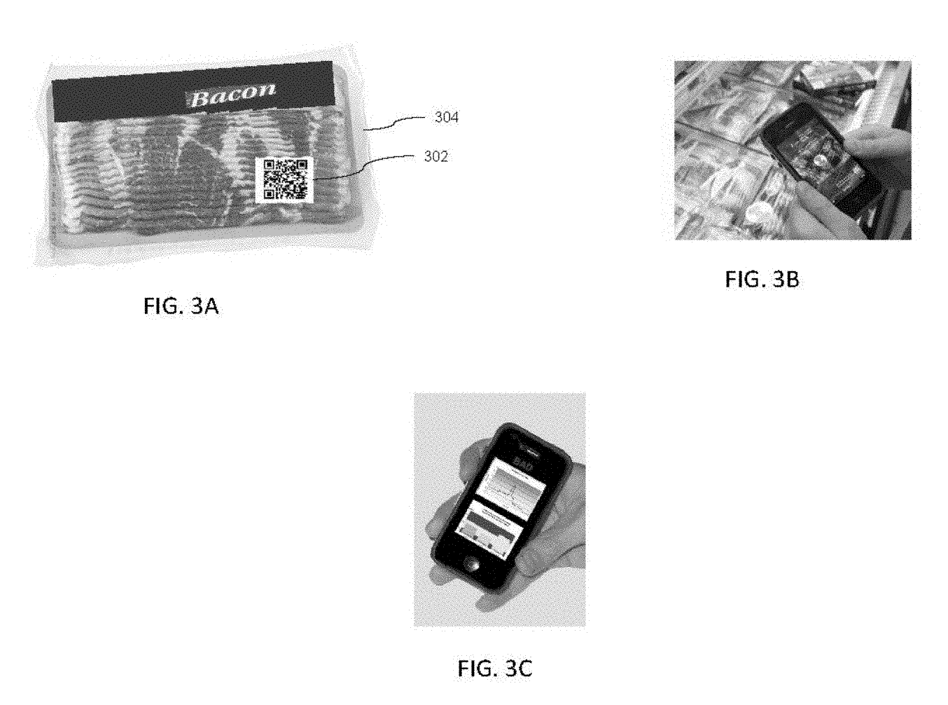

An exemplary analysis service is the Time-Temperature Pathogen Predictor (T2P2) prototype developed by the U.S. Army Natick Soldier Research Development and Engineering Center as an enhancement to the Temperature History Evaluation for Raw Meats (THERM) tool developed at the Univ. of Wisconsin (Madison). The analysis service 106 may receive temperature profiles and an identification of the associated food product, for example, as extracted by the scanning device 104 and may provide reports and an assessment of the edibility of the food product. FIGS. 2A and 2B display exemplary reports that may be provided by the analysis service 106. For example, the analysis service might provide a summary 202 and a graph 204 of the time and temperature profile extracted from the recording device. In addition, the analysis service may provide an analysis of the time and temperature profile as applied to the particular food product 206, and an assessment of the cumulative bacterial growth 208, 210. In at least one aspect, the report may include a color coded warning of the level of bacterial growth 208. FIG. 3C, referenced below, shows an exemplary simplified report that may be provided to the scanning device at a point of purchase. It should be understood that the disclosed reports are exemplary and may include any suitable reports related to the food product to which the recording device is attached, and that the recording device may be scanned and reports may be generated any time after the recording device is affixed to the food product.

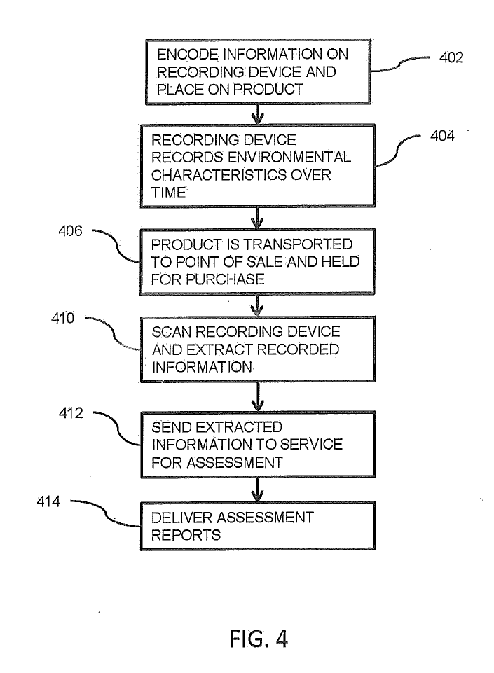

FIGS. 3A-3C in combination with FIG. 4 show exemplary illustrations and describe an exemplary procedure for implementing the disclosed embodiments. As shown in block 402 of FIG. 4 and illustrated in FIG. 3A, a recording device 302 is encoded with static information related to an identification of a food product 304 and affixed to the food product 304 at the time of manufacture or packaging. As shown in block 404, the recording device records environmental characteristics over time beginning with placement on the product. As shown in block 406, the food product 304 is then transported to a point of sale and held for purchase. As shown in block 410 and illustrated in FIG. 3B, at any time after the recording device is attached to the product, the recording device may be scanned and the recorded information, including the environmental profile may be extracted from the recording device. Block 412 shows that the extracted information is sent to the analysis service for assessment.

Exemplary information that may be recorded in the recording device may include any information related to an attached product, including any of the information described above, for example, a date of manufacture, batch numbers, suppliers, a product identifier, and a time and temperature profile, or any other information. In addition, the recorded information may include a location of the analysis service for routing the encoded information. As another example, the recording device may include a universal record locator (URL) which provides a website, a product identifier and the time and temperature profile. An exemplary URL may specify a website where the analysis service is located, for example identified in the format of a World Wide Web address or an Internet Protocol address, and may include additional information, for example, a specific manufacturer, food type and date of manufacture information, followed by a series of time points t1 . . . t45 with the temperature recorded at each of these time points. Each time point may relate to a time offset from the date of manufacture. In this example, when the URL is accessed, all the encoded information may be passed to the website specified by the web address. The information may then be parsed by the website and used as input into a food pathogen prediction model and assessment reports may be generated.

As shown in FIG. 3C and block 414 of FIG. 4, the reports shown in FIGS. 2A, 2B or 3C may be delivered to the scanning device 104. The reports in FIGS. 2A, 2B or 3C may be used at a point of purchase, or any time after the recording device is affixed to the product to determine if the food product is suitable for purchase or consumption. As shown in FIG. 3C, at least one report may include a yes or no indicator, for example, “good” or “bad.” While the reports are shown and described as being delivered to the scanning deice 104, it should be understood that the reports may be delivered to any suitable device or system.

The disclosed embodiments are directed to providing state-of-the-art food safety information directly to consumers by tracking the temperature of the food from the factory to the point of consumption by providing cost effective time and temperature recording and an ability to utilize powerful pathogen prediction models. By using programmable inks to change elements of a scannable code, implemented as a recording device, use of the scannable code has been extended to recording dynamic temperature information in the form of temperatures measured at specific time points, also referred to as a time and temperature profile. The time and temperature profile may be passed to a pathogen prediction model whenever the scannable code is scanned and the results may be immediately presented to an end user. The disclosed recording device may be produced at a cost that allows time and temperature recording for individual products, and provides a mechanism to deliver this information to a pathogen prediction model to assess food safety. The aspects of the disclosed embodiments advantageously provide science-based product safety information based on a pathogen prediction model as opposed to wide ranging estimates of food quality or safety provided by sell by dates or simple color changing stickers. The disclosed embodiments have commercial and military applications at least for temperature-sensitive, highly-perishable food products, for example, meat, poultry, dairy products, etc. and may provide food-safety-risk decision makers with valid science-based information. Retail operations that repackage food products may implement the disclosed embodiments to provide scanning at checkout to provide instantaneous product safety information to prevent the sale of potentially unsafe foods. Foodservice operations which offer temperature-sensitive prepackaged foods may also implement the disclosed embodiment in a similar manner.

The corresponding structures, materials, acts, and equivalents of all means or step plus function elements in the claims below are intended to include any structure, material, or act for performing the function in combination with other claimed elements as specifically claimed. The description of the disclosed embodiments is presented for purposes of illustration and description, but is not intended to be exhaustive or limited to the embodiments in the form disclosed. Many modifications and variations will be apparent to those of ordinary skill in the art without departing from the scope and spirit of the disclosed embodiments. The aspects of the disclosed embodiment are selected and described in order to best explain the principles of the disclosed embodiments and the practical application, and to enable others of ordinary skill in the art to understand the disclosed embodiments with various modifications as are suited to the particular use contemplated.

Thus, while there have been shown, described and pointed out, fundamental novel features of the invention as applied to the exemplary embodiments thereof, it will be understood that various omissions and substitutions and changes in the form and details of devices and methods illustrated, and in their operation, may be made by those skilled in the art without departing from the spirit of the invention. Moreover, it is expressly intended that all combinations of those elements and/or method steps, which perform substantially the same function in substantially the same way to achieve the same results, are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A system for determining food safety of a food product for human consumption by a user at a point of purchase, comprising:
a recording device printed with one or more inks configured to change characteristics in response to changes in ambient temperature conditions over predetermined time intervals, the recording device including a label being mounted to a food package containing the food product, the label having information as to the food product including an identifier of a food product, the label also including ink that varies with temperature over predetermined time periods to record an environmental profile of the food package containing the food product and the label further including a URL for an analysis service;
a portable scanning device comprising a mobile phone configured to scan the label to ascertain the identifier and the environmental profile and output the identifier and the environmental profile via a wireless link at a point of purchase;
the analysis service being configured to receive the identifier and the environmental profile via the wireless link and to analyze the environmental profile using the identifier of the food product and wherein the analysis service further uses models which predict bacteria growth in response to temperature changes over time to provide at least one assessment of whether the food product is safe for human consumption, the analysis service configured to output via the wireless link the at least one assessment of whether the food product is safe for human consumption;
wherein the at least one assessment of whether the food product is safe for human consumption includes a graph showing the food pathogen cumulative log change and color coded indication of the safety of the food pathogen cumulative log change;
wherein the mobile phone is further configured to receive the at least one assessment of whether the food product is safe for human consumption via the wireless link and indicate the assessment of whether the food product is safe for human consumption to a user.

2. A method for determining food safety of a food product for human consumption by a user at a point of purchase, comprising:
printing a recording device with one or more inks configured to change characteristics in response to changes in ambient temperature conditions over predetermined time intervals onto a food package containing the food product, the recording device including a label being mounted to the food package, the label having information as to the food product including an identifier of a food product, the label also including ink that varies with temperature over predetermined time periods to record an environmental profile of the food package containing the food product and the label further including a URL for an analysis service;
using a portable scanning device comprising a mobile phone configured to scan the label to ascertain the identifier and the environmental profile and output the identifier and the environmental profile via a wireless link at a point of purchase;
using the analysis service to receive the identifier and the environmental profile via the wireless link and to analyze the environmental profile using the identifier of the food product and wherein the analysis service further uses models which predict bacteria growth in response to temperature changes over time to provide at least one assessment of whether the food product is safe for human consumption, the analysis service configured to output via the wireless link the at least one assessment of whether the food product is safe for human consumption and wherein the at least one assessment of whether the food product is safe for human consumption includes a graph showing the food pathogen cumulative log change and color coded indication of the safety of the food pathogen cumulative log change; and
further using the mobile phone to receive the at least one assessment of whether the food product is safe for human consumption via the wireless link and indicate the assessment of whether the food product is safe for human consumption to a user.
